(19) United States
(12) Patent Application Publication
(10) Pub. No.: US 2025/0213590 A1
(43) Pub. Date: Jul. 3, 2025

(54) Combination Treatment Of Hypersensitivity Disorders

(71) Applicant: Ivig Consultants Inc., Bratenahi, OH (US)

(72) Inventor: Robert Hostoffer, Bratenahl, OH (US)

(21) Appl. No.: 18/123,092

(22) Filed: Mar. 17, 2023

(51) Int. Cl. A61K 31/573; A61K 31/4174; A61K 31/55; A61P 37/08

(52) U.S. Cl. CPC A61K 31/573; A61K 31/4174; A61K 31/55; A61P 37/08

(57) ABSTRACT

Provided are compositions and methods of treatment of allergic disorders and other hypersensitivity disorders including allergic rhinitis. For instance, provided is a method of treating a subject afflicted with an allergic disorder or a hypersensitivity disorder comprising administering to the subject a therapeutically effective amount of an antihistamine, a therapeutically effective amount of a corticosteroid and a therapeutically effective amount of a nasal decongestant, for at least four days, thereby treating the subject.

### FIELD OF THE INVENTION

The invention relates to compositions and methods of treatment of allergic disorders and other hypersensitivity disorders including allergic rhinitis. All documents cited to or relied upon below are expressly incorporated herein by reference.

### BACKGROUND OF THE INVENTION

Allergic disorders and other hypersensitivity disorders are exaggerated or inappropriate immune reactions to foreign antigens. Allergic rhinitis (AR) is a type of allergic disorder and is a chronic condition with clinical manifestations of nasal congestion, itching, pressure, sneezing, rhinorrhea, and postnasal drips that affects daily quality of life. AR has traditionally been treated with various medications. However, improved treatment methods and compositions are needed.

A known treatment strategy for allergic disorders and other hypersensitivity disorders is the administration of antihistamines. Antihistamines block histamine receptors but do not affect histamine production or metabolism. Azelastine is an antihistamine indicated for the treatment of the symptoms of seasonal allergic rhinitis and the treatment of the symptoms of vasomotor rhinitis.

Another known treatment strategy for allergic disorders and other hypersensitivity disorders is the administration of corticosteroids. Corticosteroids are anti-inflammatory drugs often known as steroids. Glucocorticoids are a type of corticosteroid. Triamcinolone is a glucocorticoid that has been used to temporarily relieve certain symptoms of hay fever or other upper respiratory allergies.

Nasal decongestants are commonly used for short-term relief of nasal congestion. Oxymetazoline, is a nasal decongestant which has been used for short-term relief of nasal congestion. However, oxymetazoline nasal spray disadvantageously has not been recommended to be used longer than 3 days and current literature teaches that prolonged use of oxymetazoline nasal sprays may result in congestion rebound, i.e., recurrent or worsening nasal congestion. Merck Manual Professional Version. Allergic Rhinitis. In fact, the current drug label for Afrin® (oxymetazoline hydrochloride spray) states “do not use for more than 3 days. Use only as directed. Frequent or prolonged use may cause nasal congestion to recur or worsen.”

Presently, there is currently no fixed recommended regimen for AR treatments. A survey in Europe has shown that 42% of AR patients use multiple drug regimens at the same time to manage symptoms. However, only 33.5% of AR patients were found to be satisfied with their current treatments. There is a need for the development of new therapies for better management of AR symptoms.

### SUMMARY OF THE INVENTION

The present invention is directed to a method of treating a subject afflicted with an allergic disorder or a hypersensitivity disorder comprising periodically administering to the subject a therapeutically effective amount of a triple combination of an antihistamine, a therapeutically effective amount of a corticosteroid and a therapeutically effective amount of a nasal decongestant, for at least four days, thereby treating the subject and surprisingly preventing rebound nasal congestion from occurring after day 3 of the administration. Most typically, a single composition is administered comprising the antihistamine, the corticosteroid and the nasal decongestant and the composition comprises a weight ratio of antihistamine to corticosteroid to nasal decongestant of 1 to 9 to 0.7-2.2.

Further. AR symptom control therapy has been widely available both as over the counter (“OTC”) medications and as prescription medications. However, more than half of AR patients are not satisfied with the current symptom management regimen. The triple combination described herein provides a cost effective and easy to use solution for AR patients to manage their symptoms for a longer period of time.

### DETAILED DESCRIPTION OF TH E INVENTION

It is to be understood that the descriptions of the present invention have been simplified to illustrate elements that are relevant for a clear understanding of the present invention, while eliminating, for the purpose of clarity, many other elements found in typical pharmaceutical compositions. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

Provided is a method of treating a subject afflicted with an allergic disorder or a hypersensitivity disorder comprising periodically administering to the subject a therapeutically effective amount of an antihistamine, a therapeutically effective amount of a corticosteroid and a therapeutically effective amount of a nasal decongestant, for at least four days, thereby treating the subject and preventing rebound nasal congestion from occurring after day three of the administration.

In certain embodiments, a single composition is administered comprising the antihistamine, the corticosteroid and the nasal decongestant.

In embodiments, the composition comprises a weight ratio of antihistamine to corticosteroid to nasal decongestant of 1 to 9 to 0.7-2.2.

In embodiments, the composition comprises a weight ratio of antihistamine to corticosteroid of 1 to 9.

In embodiments, the allergic disorder or the hypersensitivity disorder is allergic rhinitis, vasomotor rhinitis, gustatory rhinitis, occupational rhinitis, or nonallergic rhinitis with eosinophilia syndrome. In one embodiment, the allergic rhinitis is selected from seasonal allergic rhinitis and perennial allergic rhinitis.

In some embodiments, the antihistamine is cetirizine, olapatadine, azelastine, or a combination thereof.

In an embodiment, the corticosteroid is a glucocorticoid. In some embodiments, the corticosteroid is beclomethasone, budesonide, flunisolide, fluticasone, mometasone, triamcinolone, cyclosomide, or a combination thereof.

In some embodiments, wherein the nasal decongestant is oxyimetazoline and/or phenylephrine.

In some embodiments, the amounts of the antihistamine, the corticosteroid and the nasal decongestant are administered intranasally.

In further embodiments, the antihistamine, the corticosteroid and the nasal decongestant are administered daily or more often than once daily. In another embodiment, the antihistamine, the corticosteroid and the nasal decongestant are administered once every 6 hours, once every 12 hours, once every 24 hours, or twice every 24 hours.

In some embodiments, the method further comprises forming a single composition comprising the antihistamine, the corticosteroid and the nasal decongestant prior to the administering step.

In further embodiments, the periodic administration of the antihistamine, the corticosteroid and the nasal decongestant continues for at least 3 days, for at least 4 days, for at least 5 days, for at least 6 days, for at least one week, for at least 30 days.

In some embodiments, the periodic administration of the antihistamine, the corticosteroid and the nasal decongestant continues for at least 4 days and rebound nasal congestion does not occur during the periodic administration.

In an embodiment, the amount of the antihistamine when taken alone, the amount of the curticosteroid when taken alone, the amount of the nasal decongestant when taken alone, or each such amount when taken alone is not effective to treat the subject. In other embodiments, the amount of the antihistamine when taken alone, the amount of the corticosteroid when taken alone, the amount of the nasal decongestant when taken alone, or each such amount when taken alone is less effective to treat the subject.

In an embodiment, treating comprises improving one or more of nasal congestion, nasal pressure, runny nose, postnasal drip, rhinorrhea and total symptoms in the subject.

In most embodiments, the subject is a human patient.

In embodiments, nasal congestion rebound is prevented, lessened or minimized. In embodiments, the nasal congestion is compared to a comparable subject who was administered the nasal decongestant only. In other embodiments, the nasal decongestion after day 3 is compared to a comparable subject who was administered the nasal decongestant and one of the corticosteroid or the antihistamine. In embodiments, the nasal congestion does not reoccur or does not worsen after day 3 of administration, in embodiments, the nasal congestion is reduced or prevented within the first three days of the periodic administration and does not reoccur or worsen after day 3 of administration.

Also provided is a method of treating a subject afflicted with seasonal allergic rhinitis or perennial allergic rhinitis comprising periodically administering to the subject a single composition comprising a therapeutically effective amount of an antihistamine, a therapeutically effective amount of a corticosteroid and a therapeutically effective amount of a nasal decongestant, for at least four days, thereby treating the subject and preventing rebound nasal congestion from occurring after day 3 of the administration.

Provided is a method of treating a subject afflicted with an allergic disorder or a hypersensitivity disorder comprising periodically administering to the subject a therapeutically effective amount of an antihistamine, a therapeutically effective amount of a corticosteroid and a therapeutically effective amount of a nasal decongestant, thereby treating the subject.

Further provided is a kit for treating a subject afflicted with an allergic disorder or a hypersensitivity disorder, comprising a therapeutically effective amount of an antihistamine, a therapeutically effective amount of a corticosteroid, a therapeutically effective amount of a nasal decongestant and an insert comprising instructions for use of the kit.

Additionally provided is a pharmaceutical composition comprising an amount of an antihistamine, an amount of a corticosteroid and an amount of a nasal decongestant.

In embodiments, the pharmaceutical composition comprises essentially the amount of the antihistamine, the amount of the corticosteroid and the amount of the nasal decongestant. In some embodiments, the pharmaceutical composition is for use in treating a subject afflicted with an allergic disorder or a hypersensitivity disorder, wherein the amount of the amount of the antihistamine, the amount of the corticosteroid and the amount of the nasal decongestant are to be administered simultaneously, contemporaneously or concomitantly.

Further provided is a therapeutic package for dispensing to, or for use in dispensing to, a subject afflicted with an allergic disorder or a hypersensitivity disorder, which comprises: a) one or more unit doses, each such unit dose comprising: i) an amount of an antihistamine, and ii) an amount of a corticosteroid and iii) an amount of a nasal decongestant; wherein the respective amounts of said antihistamine, said corticosteroid and said nasal decongestant in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

Also provided is an antihistamine for use as an add-on therapy or in combination with a corticosteroid and a nasal decongestant in treating a subject afflicted with an allergic disorder or a hypersensitivity disorder.

Further provided herein is a corticosteroid for use as an add-on therapy or in combination with an antihistamine and a nasal decongestant in treating a subject afflicted with an allergic disorder or a hypersensitivity disorder.

Also provided is use of an amount of an antihistamine, an amount of a corticosteroid and an amount of a nasal decongestant in the preparation of a combination for treating a subject afflicted with cancer wherein the antihistamine, the corticosteroid, and the nasal decongestant are prepared to be administered simultaneously, contemporaneously or concomitantly.

Further provided is a combination of an antihistamine, a corticosteroid, and a nasal decongestant for use in the manufacture of a medicament. In embodiments, the medicament is for the treatment, prevention, or alleviation of a symptom of an allergic disorder or a hypersensitivity disorder.

Also provided is a pharmaceutical combination, comprising a therapeutically effective amount of an antihistamine, a therapeutically effective amount of a conicosteroid, and a therapeutically effective amount of a nasal decongestant.

Each embodiment disclosed herein is contemplated as being applicable to each of the other disclosed embodiments. For instance, all combinations of the various elements described herein are within the scope of the invention. Additionally, the elements recited in the packaging and pharmaceutical composition embodiments can be used in the method and use embodiments described herein and vice versa.

### Definitions

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics. 10^{th} Ed, McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The articles “a” and “an” are used in this disclosure to refer to one or more than one (i.e., to at least one) of the grammatical object of the article.

A “subject” is a human, and the terms “subject” and“patient” are used interchangeably herein.

The term “treating.” with regard to a subject, encompasses, e.g. inducing inhibition, regression, or stasis of a disease or disorder; or curing, improving, or at least partially ameliorating the disorder; or alleviating, lessening, suppressing, inhibiting, reducing the severity of, eliminating or substantially eliminating, or ameliorating a symptom of the disease or disorder. “Inhibition” of disease progression or disease complication in a subject means preventing or reducing the disease progression and/or disease complication in the subject.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

As used herein. “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times a week and so on, etc.

As used herein, a “unit dose”, “unit doses” and “unit dosage form(s)” mean a single drug administration entity/entities.

As used herein, “effective” or -therapeutically effective” when referring to an amount of an antihistamine, a corticosteroid, and/or a nasal decongestant refers to the quantity of the antihistamine, the corticosteroid, and/or the nasal decongestant that is sufficient to yield a desired therapeutic response. In certain embodiments, an effective amount refers to an amount effective, at dosages and for periods of time necessary, to achieve the desired therapeutic or prophylactic result. A therapeutically effective amount of an antihistamine, a corticosteroid, and a nasal decongestant of the invention may vary according to factors such as the disease state, age, sex, and weight of the individual, and the ability of the compound to elicit a desired response in the individual. A therapeutically effective amount encompasses an amount in which any toxic or detrimental effects of the compound are outweighed by the therapeutically beneficial effects. In one embodiment, the amount of the antihistamine, the amount of the corticosteroid, and the amount of the nasal decongestant are effective to treat the subject.

The term “disorder” is used in this disclosure to mean, and is used interchangeably with, the terms disease, condition, or illness, unless otherwise indicated.

The combination of the invention may be formulated for its simultaneous, separate or sequential administration, with at least a pharmaceutically acceptable carrier, additive, adjuvant or vehicle as described herein. Thus, the combination of the three active compounds may be administered.
as a combination that is part of the same medicament formulation, the three active compounds are then administered simultaneously, or
as a combination of three units, each with one of the active substances giving rise to the possibility of simultaneous, sequential or separate administration.

As used herein, “combination” means an assemblage of reagents for use in therapy either by simultaneous or contemporaneous administration. Simultaneous administration refers to administration of an admixture (whether a true mixture, a suspension, an emulsion or other physical combination) of an antihistamine, a corticosteroid, and/or a nasal decongestant. In this case, the combination may be the admixture or separate containers of the antihistamine, the corticosteroid, and the nasal decongestant that are combined just prior to administration. Contemporaneous administration, or concomitant administration refers to the separate administration of the antihistamine, the corticosteroid, and the nasal decongestant at the same time, or at times sufficiently close together that a synergistic activity relative to the activity of either an antihistamine alone, a corticosteroid alone, and/or a nasal decongestant alone is observed or in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding nasal decongestant therapy to a patient already receiving antihistamine, and/or corticosteroid therapy.

Any known antihistamine may be utilized in the practice of the invention, a broad variety of which are known and disclosed in the art. The antihistamine may be selected, for example, from one or a combination of cetirizine, olopatadine, and/or azelastine.

Any known corticosteroid or glucocorticoid may be utilized in the practice of the invention, a broad variety of which are known and disclosed in the art. The corticosteroid may be selected, for example, from one or a combination of beclomethasone, budesonide, flunisolide, fluticasone, mometasone, cyclisonide and/or triatmcinolone.

Any known nasal decongestant may be utilized in the practice of the invention, a broad variety of which are known and disclosed in the art. The nasal decongestant may be selected, for example, from one or a combination of oxymetazoline and phenylephrine.

### Dosage and Administration:

The compounds of the present invention may be formulated in a wide variety of nasal administration dosage forms and carriers. The administration is generally nasal using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

As used herein, “an antihistamine” means an antihistamine or a pharmaceutically acceptable salt thereof. For example, “azelastine” means azelastine or a pharmaceutically acceptable salt thereof.

As used herein, “a corticosteroid” means a corticosteroid or a pharmaceutically acceptable salt thereof. For example, “triamcinolone” means triamcinolone or a pharmaceutically acceptable salt thereof.

As used herein, “a nasal decongestant” means a nasal decongestant or a pharmaceutically acceptable salt thereof. For example, “oxynetazoline” means oxymetazoline or a pharmaceutically acceptable salt thereof.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

When an amount of a compound is disclosed herein, the amount may refer to the amount of the non-salt form or the amount of the salt form. In embodiments, the amount is the salt form.

Liquid formulations also are suitable for nasal or oral administration include liquid formulation including emulsions, syrups, elixirs, aqueous solutions, aqueous suspensions.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Suitable formulations along with pharmaceutical carriers, diluents and excipients are described in Remington: The Science and Practice of Pharmacy 1995, edited by E. W. Martin, Mack Publishing Company. 19th edition, Easton, Pennsylvania. A skilled formulation scientist may modify the formulations within the teachings of the specification to provide numerous formulations for a particular route of administration without rendering the compositions of the present invention unstable or compromising their therapeutic activity.

### EXAMPLE

The following example further describes and demonstrates particular embodiments within the scope of the present invention. The disclosure is further illustrated by the following example, which is not to be construed as limiting this disclosure in scope or spirit to the specific procedures herein described. It is to be understood that the example is provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

A triple spray (TS) combination of azelastine, triamcinolone, and oxymetazoline delivered in a single spray bottle was examined to determine if it would provide better symptom relief compared to a single spray (SS) or a double spray (DS) combination.

Two hundred patients were surveyed. Oral consents were obtained from the patients prior to their completion of the surveys. Inclusion criteria included age 18 and above, a diagnosis of seasonal or perennial allergic rhinitis (AR), and the use of at least one intranasal treatment. Data was separated into three groups: single spray, double spray, and triple spray. The SS therapy was defined as the use of one intranasal treatment. The DS therapy was defined as the use of two intranasal treatments. The TS therapy included a mixture of 10.8 ml of triamcinolone. 30 mL of 0.1% azelastine, and 8 sprays of 0.05% oxymetazoline in a single spray bottle.

The patients self-administered one spray into each nostril twice daily for each of the SS therapy, the DS therapy and the TS therapy. For patients with seasonal AR, the administration started at the start of the allergy season and continued until the end of the allergy season. For patients with perennial AR, the administration occurs throughout the year.

The survey asked patients to rate their level or nasal congestion, runny nose, itchy nose, and sneezing from none (0), mild (1), moderate (2), to severe (3) and computed an overall symptom scoring. The survey then asked how well their regimen controlled their congestion, pressure, runny nose, and postnasal drip with the same rating scale and computed an overall symptom control score. The differences between groups were determined by comparing the mean differences in each symptom severity category and symptom control category among the three groups.

Out of the 200 surveyed, 93 patients used the SS, 71 patients used DS, and 36 patients used TS. Patients who used SS used either an intranasal antihistamine or an intranasal glucocorticoids treatment. Patients who used the DS used a combination of an intranasal antihistamine and an intranasal glucocorticoid.

There was no statistical difference in the severity of symptoms between three groups: SS, DS, and TS. Significant differences were found in symptom control scores between the DS and SS groups in all categories: nasal congestion control (p=0.005), pressure control (p=0.003), runny nose control (p=0.001), postnasal drip control (p=0.008), total symptom control (p=0.001)(Table 1).

TABLE 1
Means score comparison between single spray and double spray,
single spray and triple spray, double spray and triple spray groups.

| | Total | Nasal | | Runny | Postnasal | Total |
|---|---|---|---|---|---|---|
| | severity | congestion | Pressure | nose | drip | symptom |
| | score | control | control | control | control | control |
| SS | 5.88 | 3.49 | 2.99 | 3.05 | 2.85 | 12.45 |
| DS | 6.29 | 3.93 | 3.51 | 3.72 | 3.32 | 14.38 |
| p | 0.175 | 0.005* | 0.003* | 0.001* | 0.008* | 0.001* |
| value | | | | | | |
| SS | 5.88 | 3.49 | 2.99 | 3.05 | 2.85 | 12.45 |
| TS | 6.08 | 3.83 | 3.53 | 3.50 | 3.08 | 13.78 |
| p | 0.347 | 0.103 | 0.024* | 0.047* | 0.182 | 0.049* |
| value | | | | | | |
| DS | 6.29 | 3.93 | 3.51 | 3.72 | 3.32 | 14.38 |
| TS | 6.08 | 3.83 | 3.53 | 3.50 | 3.08 | 13.78 |
| p | 0.339 | 0.359 | 0.469 | 0.207 | 0.181 | 0.227 |
| value | | | | | | |

SS = single spray;
DS = double spray;
TS = triple spray
*Statistically significant p values

When comparing SS and TS, there was a significant difference in symptom control in pressure control (p=0.024) and runny nose control (p=0.47) and overall total symptom control score (p=0.049) (Table 1). There was no significant difference between the mean symptom control scores between DS and TS in all categories (Table 1).

When patients' symptoms are not controlled with intranasal glucocorticoids alone, adding an oral antihistamine or an intranasal antihistamine is recommended for severe AR. Topical antihistamine has been shown to be more effective than oral antihistamine in managing nasal obstruction symptoms. Topical decongestants such as oxymetazoline are α-sympathomimetics that may decrease nasal mucosal swelling via activation of al receptors on the mucosa resulting in vasoconstriction of blood vessels.

The TS combination is easy for patients to mix at home in a single spray bottle. Triamcinolone is available over the counter (OTC) in a bottle that is easy to open for mixing when compared to the unremovable cap of fluticasone. The aqueous nature of triamcinolone allows easy mixing with azelastine. The addition of eight sprays of oxymetazoline provides better management of pressure symptoms. The label for triamcinolone acetonide nasal spray (Nasacort®) states that about 0.18 ml of solution is administered in each spray. Since patients used the triamcinolone bottle for administration, a total of 0.28 ml is administered into each nostril each day. The following chart shows the amounts of each of triamicinolone, azelastine, and oxynetazonline administered for each spray into the nostril.

| | Total | Percent by | | Density of | Administered |
|---|---|---|---|---|---|
| | Volume in | volume in | | active | in each spray |
| | mixture (ml) | mixture | ml per spray | (mg/ml) | (mg) |
| Triamicinolone | 10.8 | 26.1627907 | 0.047093023 | 0.305555556 | 0.014389535 |
| Azelastine | 30 | 72.6744186 | 0.130813953 | 1 | 0.130813953 |
| Oxynetazonline | 0.48 | 1.162790698 | 0.002093023 | 0.5 | 0.001046512 |
| (min) | | | | | |
| Total (min) | 41.28 | | | | |
| Triamicinolone | 10.8 | 19.56521739 | 0.035217391 | 0.305555556 | 0.01076087 |
| Azelastine | 30 | 54.34782609 | 0.097826087 | 1 | 0.097826087 |
| Oxynetazonline | 14.4 | 26.08695652 | 0.046956522 | 0.5 | 0.023478261 |
| (min) | | | | | |
| Total (max) | 55.2 | | | | |

*The min and the max represemt the range of administration amounts of oxynetazonlineprovided by Hakim 2016.

The following chart shows the amounts of each of triamicinolone, azelastine, and oxynetazonline administered for each two sprays into the nostril compared to the label recommend amounts to be administered. As shown below, the amounts of each of triamicinolone, azelastine, and oxynctazonline are lower than the amounts recommended to be administered in each of their respective labels. Yet, surprisingly, the administration showed clear benefits despite the reduction in amounts administered.

| | Administered in | Label suggested |
|---|---|---|
| | two sprays (g) | administration (mg) |
| Triamicinolone | 0.02877907 | 0.11 |
| Azelastine | 0.261627907 | 0.274 |
| Oxynetazonline | 0.002093023 | 0.005 |
| (min) | | |
| Triamicinolone | 0.021521739 | 0.11 |
| Azelastine | 0.195652174 | 0.274 |
| Oxynetazonline | 0.046956522 | 0.245 |
| (max) | | |

This study demonstrated that TS has a significant benefit over SS in managing pressure, rhinorrhea, and overall AR symptoms. The DS combination of antihistamines and glucocorticoids have clear benefits over the SS in managing AR with similar severity. Previous studies have also demonstrated the effectiveness of a DS of azelastine and fluticasone delivered in a single spray bottle for management of AR symptoms where monotherapy of either antihistamine or glucocorticoids does not adequately manage symptoms. Although there is no difference in AR management between the DS and TS combinations in this study, the TS delivered in a single spray bottle may be more convenient to use when compared to the administration of two different nasal spray treatments. Although both TS and DS combinations show no statistical difference in total symptom score, the only available DS combination in one bottle requires a prescription and is frequently more financially costly. Only SSs are available over the counter. The triple sprays involve two OTCs and one prescription medicine that is generic.

AR symptom control therapy has been widely available both as OTC medications and as prescription medications. However, more than half of AR patients are not satisfied with the current symptom management regimen. The TS combination described herein provides a cost effective and easy to use solution for AR patients to manage their symptoms. This study shows that the TS is effective in managing pressure, rhinorrhea, and overall AR symptoms compared to monotherapy of either antihistamine or glucocorticoids, but no difference in AR symptom control when compared to the use of two spray combination. The fixed combination of azelastine and fluticasone delivered in a single spray bottle has established evidence in favor of its effectiveness, but the current cost of this medication is higher than the homemade TS combination which imposes a long-term financial burden.

### REFERENCES

Brożek XL, Bousquet J. Agache 1, et al Allergic Rhinitis and its Impact on Asthma (ARIA) guidelines-2016 revision. J Allergy Clin. Immunol. 2017; 140(4):950-958. doi:10.1016/j.jaci.2017.03.050.
Bjermer L. Westman M. Holmstrom N, Wickman MC. The complex pathophysiology of allergic rhinitis: scientific rationale for the development of an alternative treatment option. Allergy Asthma Clin Immunol. 2019:15:24. 16 doi:10.1186s13223-018-0314-1.
Klimek L. Sped A, Becker S. Mösges R, Tomazic PV. Current therapeutical strategies for allergic rhinitis. Expert Opin Pharmacother. 2019:20(1):83-89. doi:10.1080/14656566.2018.1543401
Klimek L, Sped A. Evidenzbasierte Therapicoptionen von Allergien im HNO-Bereich: Ein Update [Evidence-based treatment options for allergic diseases in otolaryngology: an update]. HNO. 2013; 61(6):525-538. doi:10.1007s00106-013-2709-0
Wise SK, Lin SY, Toskala E. et al. International Consensus Statement on Allergy and Rhinology: Allergic Rhinitis. Int Forum Allergy Rhinol. 2018; 8(2):108-352, doi:10.1002/alr.22073.
Canonica GW, Bousquet J, Mullol J, Scadding GK, Virchow JC. A survey of the burden of allergic rhinitis in Europe. Allergy. 2007; 62 Suppl 85:17-25, doi:10.1111/j.1398-9995.2007.01549.x
Ciprandi G, incorvaia C, Scurati S, et al. Patient-related factors in rhinitis and asthma: the satisfaction with allergy treatment suvey. Curr Med Res Opin. 2011; 27(5):1005-1011. doi:10.1185/03007995.2011.559580.
Hampel FC, Ratner PH, Van Bavel J, et al. Double-blind, placebo-controlled study of azelastine and fluticasone in a single nasal spray delivery device Ann Allergy Asthma Immunol. 2010; 05(2):168-173. doi:10.1016/j.anai.2010.06.008.
Carr W, Bernstein J, Liebernian P, et al. A novel imranasal therapy of azelastine with fluticasone for the treatment of allergic rhinitis. J Allergy Clin Immunol. 2012; 129(5):1282-1289.e10. doi:10.10116/j.jaci.2012.01.077.
Neighbors CL. Salvador CF. Zhu B. Camacho M, Tsai P. Intranasal corticosteroid and oxymetazoline for chronic rhinitis: a systematic review. J Laryngol Otol. 2022 January; 136(1):8-16. doi: 10.1017/0022215121003364. Epub 2021 Oct. 27. PMID: 34702392.
Merck Manual Professional Version, Allergic Rhinitis, accessed Feb. 22, 2023, available at: www.merckmanals.com/professional/immunology-atlergic-disorder/allergic,-autoimmune,-and-other-hypersensitivity-disorders/allergic-rhinitis; Hakim M, Walia H, Rafiq M, Grannell T. Cartabuke RS, Tobias JD. Oxymetazoline Metered Dose Spray: Factors Affecting Delivery Volume. J Pediatr Pharmacol Ther. 2016 May-Jun.; 21(3):247-51. doi: 10.5863/1551-6776-21.3.247. PMID: 27453703; PMCID: PMC4956333.

1. A method of treating a subject afflicted with an allergic disorder or a hypersensitivity disorder comprising periodically administering to the subject a therapeutically effective amount of an antihistamine, a therapeutically effective amount of a corticosteroid and a therapeutically effective amount of a nasal decongestant, for at least four days, thereby treating the subject and preventing rebound nasal congestion from occurring after day three of the administration, wherein the composition comprises a weight ratio of antihistamine to corticosteroid to nasal decongestant of 1 to 9 to 0.7-2.2.

2. The method according to claim 1, wherein a single composition is administered comprising the antihistamine, the corticosteroid and the nasal decongestant.

3. A method of treating a subject afflicted with comprising periodically administering to the subject a therapeutically effective amount of an antihistamine, a therapeutically effective amount of a corticosteroid and a therapeutically effective amount of a nasal decongestant, thereby treating the subject.

4. The method of claim 1, wherein the allergic disorder or the hypersensitivity disorder is allergic rhinitis, vasomotor rhinitis, gustatory rhinitis, occupational rhinitis, or nonallergic rhinitis with eosinophilia syndrome.

5. The method of claim 1, wherein the allergic disorder or the hypersensitivity disorder is an allergic rhinitis selected from seasonal allergic rhinitis and perennial allergic rhinitis.

6. The method according to claim 1, wherein the antihistamine is cetirizine, olapatadine, azelastine, or a combination thereof.

7. The method according to claim 1, wherein the corticosteroid is a glucocorticoid.

8. The method according to claim 1, wherein the corticosteroid is beclomethasone, budesonide, flunisolide, fluticasone, mometasone, triamcinolone, cyclosomide, or a combination thereof.

9. The method according to claim 1, wherein the nasal decongestant is oxymetazoline and/or phenylephrine.

10. The method according to claim 1, wherein the amounts of the antihistamine, the corticosteroid and the nasal decongestant are administered intranasally.

11. The method according to claim 1, wherein the antihistamine, the corticosteroid and the nasal decongestant are administered daily or more often than once daily.

12. The method according to claim 1, wherein the antihistamine, the corticosteroid and the nasal decongestant are administered once every 6 hours, once every 12 hours, once every 24 hours, or twice every 24 hours.

13. The method of claim 1, further comprising forming a single composition comprising the antihistamine, the corticosteroid and the nasal decongestant prior to the administering step.

14. The method according to claim 1, wherein the periodic administration of the antihistamine, the corticosteroid and the nasal decongestant continues for at least 5 days, for at least 6 days, for at least one week, for at least two weeks, for at least 30 days.

15. The method according to claim 1, wherein treating comprises improving one or more of nasal congestion, nasal pressure, runny nose, postnasal drip, rhinorrhea and total symptoms in the subject.

16. The method according to claim 1, wherein rebound nasal congestion does not occur during the periodic administration.

17. The method according to claim 1, wherein the amount of the antihistamine when taken alone, the amount of the corticosteroid when taken alone, the amount of the nasal decongestant when taken alone, or each such amount when taken alone is not effective to treat the subject.

18. The method according to claim 1, wherein the amount of the antihistamine when taken alone, the amount of the corticosteroid when taken alone, the amount of the nasal decongestant when taken alone, or each such amount when taken alone is less effective to treat the subject.

19. The method according to claim 1, wherein the subject is a human patient.

20. A kit for treating a subject afflicted with an allergic disorder or a hypersensitivity disorder, comprising a therapeutically effective amount of an antihistamine, a therapeutically effective amount of a corticosteroid, a therapeutically effective amount of a nasal decongestant and an insert comprising instructions for use of the kit.

21. A method of treating a subject afflicted with an allergic disorder or a hypersensitivity disorder, comprising periodically administering to the subject a triple spray mixture of 10.8 mL of triamcinolone, 30 mL of 0.1% azelastine, and 8 sprays of 0.05% oxymetazoline, for at least four days, thereby treating the subject and preventing rebound nasal congestion from occurring after day three of the administration.

22. A kit for treating a subject afflicted with an allergic disorder or a hypersensitivity disorder, comprising a spray bottle containing a mixture of 10.8 mL of triamcinolone, 30 mL of 0.1% azelastine, and 8 sprays of 0.05% oxymetazoline, and an insert comprising instructions for use of the kit.
